(19) United States
(12) Patent Application Publication
(10) Pub. No.: US 2012/0116163 A1
(43) Pub. Date: May 10, 2012

(54) CONTROL DEVICE

(75) Inventors: Theodor Lutze, Balgheim (DE); Olaf Hegemann, Tuebingen (DE)

(73) Assignee: Aesculap AG, Tuttlingen (DE)

(21) Appl. No.: 13/304,818

(22) Filed: Nov. 28, 2011

Related U.S. Application Data
(63) Continuation of application No. PCT/EP2010/055281, filed on Apr. 21, 2010.

(30) Foreign Application Priority Data
May 29, 2009 (DE) 10 2009 024 238
Sep. 14, 2009 (DE) 10 2009 042 488

(51) Int. Cl. A61B 1/00

(52) U.S. Cl. 600/118

(57) ABSTRACT

A control device is provided, in particular for use in endoscopes or the like. The control device has proximal and distal end sections each comprising an area of articulation and a central section arranged therebetween. The control device also comprises outer and inner hollow cylindrical shafts and a control element arranged between the shafts. Two or more longitudinal elements extend substantially from the proximal to the distal end section and transfer force. For an optimized control function, the longitudinal elements are arranged at essentially regular angular distances in a circumferential direction of the control device and are connected to one another in the circumferential direction in the region of their respective proximal and distal ends. The distal ends of the longitudinal elements are secured in the circumferential direction in angular positions which are different to the angular positions, in which the respectively associated proximal ends are secured.

This application is a continuation of international application number PCT/EP2010/055281 filed on Apr. 21, 2010 and claims the benefit of German application number 10 2009 024 238.4 filed on May 29, 2009 and German application number 10 2009 042 488.1 filed on Sep. 14, 2009.

The present disclosure relates to the subject matter disclosed in international application number PCT/EP2010/055281 of Apr. 21, 2010 and German applications number 10 2009 024 238.4 of May 29, 2009 and number 10 2009 042 488.1 of Sep. 14, 2009, which are incorporated herein by reference in their entirety and for all purposes.

### BACKGROUND OF THE INVENTION

The invention relates to a control device for precision mechanical or surgical applications, for example for use in endoscopes or the like.

The invention relates, in particular, to a control device for instruments for extremely exact mechanical applications or surgical applications in the minimally invasive field.

Such control devices are known from the state of the art and have a proximal end section, i.e. facing the user/surgeon, and a distal end section facing away from him, each of which comprises an area of articulation, as well as a central section which is arranged between the end sections and is often designed to be flexurally rigid. They comprise, in addition, an outer hollow cylindrical shaft, an inner hollow cylindrical shaft as well as a control element which is arranged between these shafts and has two or more longitudinal elements which extend substantially from the proximal to the distal end section of the control device and transfer force. The force transferring longitudinal elements are arranged essentially regularly in circumferential direction of the control device and are connected to one another in circumferential direction in the region of their respective proximal and distal end sections. Traction and pressure forces, with which a pivoting movement at the proximal end section may be converted into a corresponding pivoting movement at the distal end section, may be transferred via the longitudinal elements.

Control devices of this type are known, for example, from WO 2005/067785 A1, with which a plurality of force transferring longitudinal elements are used in the form of wires or cables which are arranged so as to abut directly on one another in circumferential direction and thus guide one another laterally. The outer and the inner hollow cylindrical shafts are provided for the guidance of the force transferring longitudinal elements in a radial direction and so guidance of the force transferring longitudinal elements is ensured in every direction.

A gripping element which can be actuated by hand is normally mounted on the proximal end of the control device and can, of course, also be replaced by motor driven operating elements while tools, cameras, lighting elements and the like can be connected to the distal end which is also called head.

Complex interior spaces in the mechanical field which are difficult to access, for example engines, machines, radiators and the like, may be inspected and repaired or, however, the operations in the minimally invasive field mentioned above can be carried out with such instruments containing the control device.

Control devices known thus far generate a movement of the distal end section with a respectively opposite direction of pivoting which is, in addition, also restricted to the same plane of pivoting.

Even when pivoting movements in many different directions are possible with some systems, this principle of the deflection of the distal end in an opposite direction to the deflection of the proximal end in the same plane is retained.

With a whole series of applications both in the mechanical and in the medical field, a movement at the proximal end is subject to specific spatial limits and so these control devices cannot always be used in an optimum manner.

The object of the invention is to remedy this problem.

### SUMMARY OF THE INVENTION

In this connection, the invention suggests that the distal ends of the longitudinal elements of the control device according to the invention be secured in circumferential direction in angular positions which differ from the angular positions, in which the respectively associated proximal ends are secured.

Depending on the application, it is conceivable for a set of control elements to be present for the control device, with which the difference in the angular positions of the ends of the force transferring longitudinal elements varies in circumferential direction.

Deviations of the angular positions in the circumferential direction, from which an additional benefit in the handling is to be expected, begin at approximately 10° and reach as far as approximately 350°.

In particular, differences in the angular positions at the proximal and distal end sections in the range of approximately 45° to approximately 315° are of interest, even more preferred in the range of approximately 150° to approximately 210°.

Control devices of the present invention, with which the angular positions have a difference of approximately 180°, are of particular relevance and so a mirror image movement of the proximal and distal end sections in one plane can be generated.

In one of the preferred embodiments of the control device according to the invention it is provided for the force transferring longitudinal elements of the control element to be arranged so as to be laterally spaced from one another.

In order to stabilize the force transferring longitudinal elements, which are laterally spaced, in their circumferential position, it may be provided for spacer elements to be arranged between the force transferring longitudinal elements. These may be secured to one of the shafts in the form of, for example, guiding eyelets.

It is, however, also conceivable for additional longitudinal elements, which are merely arranged between the force transferring longitudinal elements and act as spacer elements, to be present between the force transferring longitudinal elements.

Alternatively, it may also be provided for the longitudinal elements to be arranged along the longitudinal direction at least partially in direct contact with one another, wherein a multiple, essentially punctiform contact between the longitudinal elements is often sufficient to stabilize them in a lateral direction, i.e. in circumferential direction.

In the case of preferred control devices of the present invention, the longitudinal elements are guided by the outer and the inner shafts in a radial direction such that irrespective of whether the longitudinal elements are arranged so as to be laterally spaced or are in direct contact with one another partially or over the entire length, a sufficient stabilization of their geometry is provided in order to ensure an exact angle for the transfer of force from the proximal to the distal end section.

The arrangement of the longitudinal elements in circumferential direction for achieving the different angular positions at the proximal and distal ends can be brought about in various ways.

In a first variation, the force transferring longitudinal elements are arranged in a helical shape between the shafts over at least part of their entire length.

In one preferred embodiment, the force transferring longitudinal elements are arranged in a helical shape between the shafts over their entire length. In this case, with respect to the typical length of the control device of 10 cm and considerably more and with a typical diameter of a few millimeters, this results in an extremely high pitch of the helical line shape or, expressed differently, a very slight deviation from the parallelism in relation to the longitudinal direction of the control device which amounts to a few degrees of angle up to a fraction of a degree of angle.

In a further alternative, it is provided for the force transferring longitudinal elements to be arranged essentially parallel to the longitudinal direction of the control device in the region of the proximal or distal ends and to be arranged in a helical shape in a region located therebetween.

In a further variation, it is provided for the force transferring longitudinal elements to have one or more sections which are arranged parallel to the control device in the region between their proximal and distal ends, wherein other sections, in particular the proximal and distal ends, are arranged in a helical shape.

Although, in the case of the last two variations, only part of the entire length of the control element is available for achieving the angular offset, only slight angular deviations from the longitudinal direction are still necessary.

In accordance with one variation of the control device according to the invention, the force transferring longitudinal elements are designed as cables or wires.

In another variation, the force transferring longitudinal elements have a banana-shaped cross section.

In one preferred embodiment of the invention, the control device has a control element which comprises a hollow cylindrical component, the cylinder wall of which is subdivided at least in the region of a section between the proximal and distal ends into two or more wall segments which form the force transferring longitudinal elements.

In this respect, the two or more wall segments can be connected fixedly to one another at the distal end of the hollow cylindrical component via an annular collar.

In addition, the two or more wall segments can be connected fixedly to one another in the region of the proximal end of the hollow cylindrical component.

It is particularly preferred to have the hollow cylindrical component designed in one piece. In this case, the handling during assembly of the control device is particularly simple. Moreover, the one-piece component may be produced with particular precision with respect to the mutual alignment of the wall segments.

Control devices with this configuration have, in particular, a hollow cylindrical component which is manufactured from a single small tube, wherein the subdivision of the cylinder wall into wall segments is preferably brought about by means of laser beam cutting.

Control devices of this type may be realized, in addition, with very small outer diameters, for example approximately 2 mm or less, in particular approximately 1.5 mm, as well, and, nevertheless, an adequately large lumen remains in the interior, via which additional functions can be realized. For example, the lumen is still sufficient to enable the transport of pieces of tissue away from the operating area, in particular by suction, or for bringing a light source and associated optical devices to the operating area.

The control devices according to the invention are, of course, also possible with arbitrarily large diameters.

Steel alloys or nitinol lend themselves, in particular, as material for the production of the control device, in particular of the control element in the form of the hollow cylindrical component.

In one particularly preferred embodiment, the cylinder wall is slit over the greatest part, in particular more or less over the entire length in axial direction for the purpose of forming the force transferring longitudinal elements. The longitudinal elements are formed, in this respect, by cylinder wall segments which have an arc shape in cross section.

The wall segments preferably have in cross section an arc shape which corresponds to an arc angle of approximately 20° or more, in particular 30° or more.

The number of wall segments is preferably in the range of 4 to 16, even more preferred in the range of 6 to 12.

The distance of the wall segments from one another in circumferential direction (corresponds to the width of the slit) is, measured in degrees of angle, preferably approximately 2° to 15°, even more preferred approximately 4° to approximately 8°.

The width of the slit, which results during the laser beam cutting, can be increased as required and so the remaining strip-like wall segments can be moved relative to one another without contact. On account of the circular segment-like cross sections of the longitudinal elements, the contact-less state of the longitudinal elements is also retained in the case of the traction or pressure tensioning even in the areas of articulation; this applies, in particular, for a guidance of the longitudinal elements in a radial direction between an inner and an outer shaft.

The two end areas of the hollow cylindrical element remain without any slit and so the longitudinal elements remain connected to one another via annular collars.

The proximal and distal areas of articulation of the control device can be realized in different ways.

The areas of articulation of the outer and/or inner shaft preferably have several slits which extend in circumferential direction and are separated from one another in circumferential direction or rather axial direction by wall areas.

Small tubes designed in one piece can also be used for the outer and inner shafts, respectively.

Together with a control element produced from a small one-piece tube, as already described above, a very thin-walled and, nevertheless, mechanically stressable structure results in the simplest case which consists of three small tubes pushed into one another with the functions outer shaft, control element and inner shaft, wherein a device put in place by means of the control device, for example a gripping element, can be operated and positioned without any “overtalk” of the movement of the one element onto the other element resulting. In particular, a gripping element can, for example, be guided and turned within the control device without the pivoting angle and the position of the control element itself thereby being altered or the gripping function as such being affected. Counter movements will be brought about just as little; rotational movements through 360° are possible without any problem.

In addition, these control devices can easily be taken apart, sterilized and reassembled.

A respective wall section preferably has two or more, in particular three or more, slits arranged one behind the other in circumferential direction. The slits are preferably arranged in circumferential direction at equal distances from one another.

In an axial direction, the areas of articulation of preferred control devices have three or more slits arranged next to one another, wherein the slits arranged next to one another are preferably arranged so as to be offset relative to one another in circumferential direction. The distances, at which the slits are arranged in an axial direction so as to be spaced from one another, may be equal or vary, wherein the articulation properties, in particular the bending radius, can be influenced hereby.

Typically, it will be provided for the slits to be slits penetrating the cylinder wall completely. Good bending properties may, however, also be achieved when the slits do not penetrate the wall of the shaft completely but rather end, in particular, before reaching the inner circumference. As a result, the wall of the shaft remains complete as a whole which can be desirable in some applications, in particular in the case of the outer shaft.

One preferred geometry of the slits is present when the wall surfaces delimiting the slits are arranged at an acute angle relative to the radial direction. In this respect, wall surfaces of the same slit which are located opposite one another will preferably be arranged in mirror image so that a greater slit width results at the outer circumference of a shaft than adjacent to the inner circumference.

Slits which are spaced from one another in axial direction will preferably be arranged in circumferential direction so as to overlap but be offset relative to one another so that a regular arrangement of the slits results.

The wall surfaces of the slits can be inclined relative to the axial direction at an angle which deviates from 90° so that the width of the slits at the outer circumference is greater than at the inner circumference of the outer shaft. As a result, sufficiently large pivoting angles may be realized even with small slit widths without the number of slits needing to be increased or the region of articulation needing to extend over a greater axial length.

According to one variation, the inner and/or the outer shaft has a proximal and a distal section of articulation in the region of the proximal and distal areas of articulation of the control device. At least the outer shaft will preferably comprise proximal and distal sections of articulation.

Typically, the control device is designed to be flexurally rigid in its central section.

According to one embodiment of the invention, at least one of the outer and inner shafts is equipped in the longitudinal area between the proximal and distal areas of articulation with a flexurally rigid section which realizes the bending rigidity of the central section of the control device.

Whereas, in many cases, the proximal and the distal areas of articulation are designed the same and, in particular, have an equal extension in longitudinal direction of the control device, this is not absolutely necessary.

It may, in particular, be provided for the proximal and the distal areas of articulation to be of a different design, in particular also be designed with different lengths, so that a corresponding pivoting movement of the proximal area of articulation results in a smaller or intensified pivoting movement of the distal end section.

It may be provided, in particular, for the pivoting movement of the proximal and/or distal areas of articulation to be adjustable. This can be brought about, for example, in that the extension of the proximal and/or the distal area of articulation will be varied and, therefore, the pivoting behavior of the two areas of articulation relative to one another will be altered.

It may be provided, in particular, for the control device to comprise a holding device, with which parts of one of the areas of articulation can be fixed in position in a flexurally rigid manner with respect to the central section of the control device or a functional unit adjoining its proximal or distal end section.

In one variation of the control device according to the invention, the holding device can comprise a flexurally rigid sleeve which is displaceable parallel to the longitudinal axis of the central section which is, in this case, designed to be flexurally rigid.

Depending on the position of the sleeve in longitudinal direction relative to the central section, the proximal and/or distal end section and the area of articulation provided there can be influenced in their length and be influenced in their pivoting behavior.

In this respect, the flexurally rigid sleeve will preferably be arranged on the outer circumference of the flexurally rigid shaft so that the lumen of the control device remains unaffected. If the lumen of the control device is intended to be sufficiently large for specific applications, a flexurally rigid sleeve can, of course, also be arranged in the interior of the lumen. The displaceability and, in particular, also the securing in position of the flexurally rigid sleeve are, however, easier to realize when this is arranged on the outer circumference of the outer shaft.

In accordance with another variation, the holding device can comprise a supporting holding element on the functional unit which is coupled to the proximal or distal end of the control device. In this way, the area of articulation can be influenced in its pivoting behavior from the distal or proximal end side.

In accordance with a further variation of the control device according to the invention, the holding device can be positioned and, in particular, also secured in a predetermined position. As a result, it is possible to adjust in advance or readjust the pivoting behavior of distal and proximal end sections relative to one another in a manner which can be repeated and exactly predetermined.

In accordance with a further variation of the control device according to the invention, it is provided for at least one of the areas of articulation to be of an elastic design so that when the forces introduced for the pivoting of the end sections cease to act the control device will return again to its original, straight position.

These and other advantages of the invention will be explained in greater detail in the following on the basis of the drawings.

### BRIEF DESCRIPTION OF THE DRAWINGS

FIG. 1 shows the construction of a control device according to the state of the art;

FIG. 2 shows a control device of the state of the art according to FIG. 1 in an angled state;

FIG. 3 shows an overall view of a control device according to the invention;

FIGS. 4A and B show two variations of a first embodiment of a control element of a control device according to the invention;

FIGS. 5A and B show two variations of a second embodiment of a control element of a control device according to the invention;

FIGS. 6A and B show two variations of a third embodiment of a control element of a control device according to the invention;

FIGS. 7A and B show a cross section through a preferred control element or rather a preferred control device of the invention;

FIGS. 8A and B show detailed views of preferred variations of the inner and outer shafts of a control device according to the invention;

FIG. 9 shows an overall view of a further control device according to the invention; and

FIG. 10 shows an overall view of a further control device according to the invention.

### DETAILED DESCRIPTION OF THE INVENTION

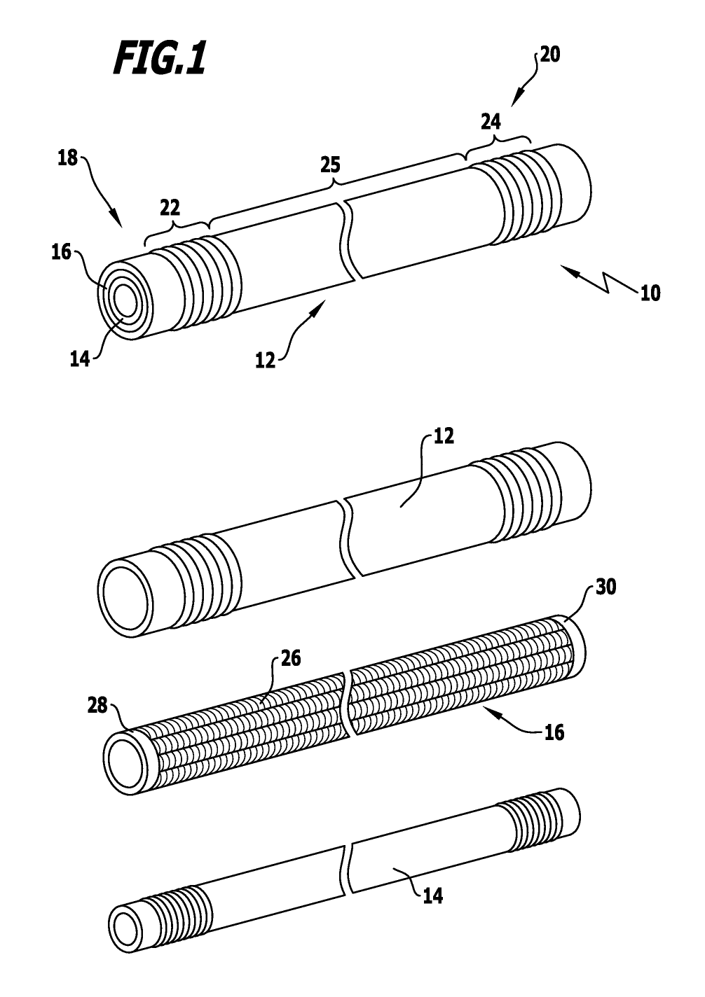

FIG. 1 shows the construction of a control device 10, as known from the state of the art, for example WO 2005/067785 A1.

In this respect, the control device 10 comprises an outer hollow cylindrical shaft 12, an inner hollow cylindrical shaft 14 as well as a control element 16 arranged between these shafts.

The outer and the inner shafts 12, 14 as well as the control element 16 have essentially the same length and are dimensioned with respect to their outer and inner diameters or wall thicknesses such that the control element can be pushed into the outer shaft with an exact fit and the inner shaft 14 into the interior of the control element 16 with an exact fit. The interior of the inner shaft 14 remains as lumen free for the introduction of instrument controls, feed lines to a camera or other optical elements and the like. The control element 16 is guided in a radial direction by the walls of the outer and the inner shafts 12, 14.

The control device 10 has a proximal end section 18 as well as a distal end section 20 which each comprise an area of articulation 22 and 24, respectively.

Typically, the area of articulation 22, 24 will be formed by a corresponding configuration of the outer and/or inner shaft 12, 14, wherein manifold suggestions for this are found in the state of the art, inter alia also in WO 2005/067785 A1.

In FIG. 1, the areas of articulation 22, 24 are merely indicated in the form of bellows-like structures.

In FIGS. 1a, 1b and 1c, the individual elements of the control device 10 of FIG. 1 are illustrated again, wherein FIG. 1a represents the outer shaft 12, FIG. 1b the control element 16 and FIG. 1c the inner shaft 14.

The outer shaft 12 has, in the regions which correspond to the areas of articulation 22 and 24, a structure which ensures the flexibility or pliability of the outer shaft 12 in this area. For example, bellows-like structures can be used in this case, as mentioned above. Alternatively, the corresponding pliability or flexibility can also be provided by a weakening of the wall of the outer shaft 12 in the sections corresponding to the areas of articulation 22, 24.

The inner shaft 14 in FIG. 1c can have a similar structure to the outer shaft 12 in FIG. 1a and so reference can be made to the description of FIG. 1a.

The control element 16 of FIG. 1b comprises a plurality of, in the present example eight, force transferring longitudinal elements which are arranged parallel to the longitudinal direction of the control element 16 and which are connected laterally to one another in circumferential direction to form annular collars 28, 30 at the respective ends of the control element 16.

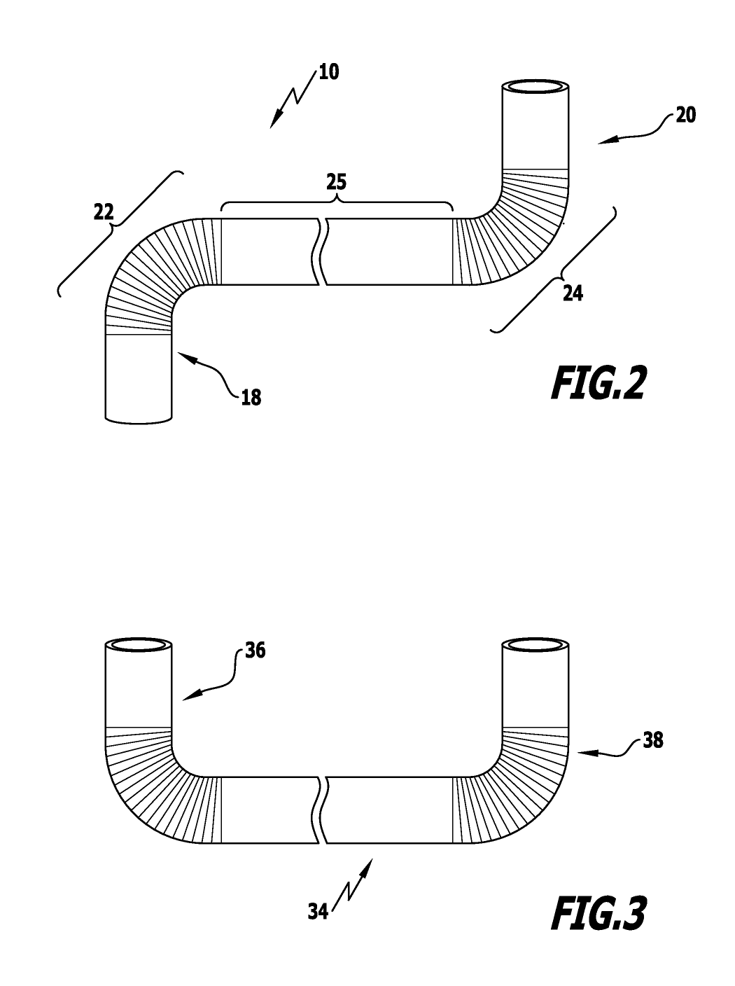

On account of the guidance of the force transferring longitudinal elements 26 between the outer and the inner shaft 12, 14 in the control device 10, any pivoting of the proximal end section 18 results in an angling at the distal end section in the region of the area of articulation 24 by the same angular amount in the same plane of pivoting but in an opposite direction. Such a situation is illustrated in FIG. 2.

In contrast hereto, it is possible with the control device according to the invention to carry out pivoting of the distal section of articulation in different arbitrarily predeterminable directions with respect to the pivoting movement of the proximal end, also in directions which are not located in the same plane.

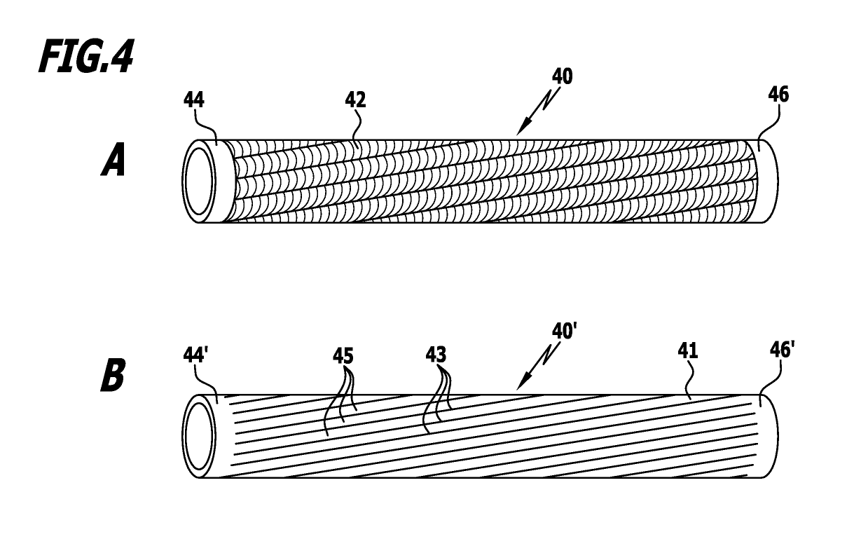

One example for this is shown in FIG. 3 on the basis of a control device 34 according to the invention, the control elements of which will be discussed in the following on the basis of FIGS. 4, 5 and 6 and are designed in accordance with the invention and, when, for example, the proximal section 36 performs a pivoting movement upwards, likewise bring about a pivoting movement of the distal section 38 upwards in the same plane.

In the case of the control elements designed in accordance with the invention, the force transferring longitudinal elements are secured in circumferential direction with their proximal and distal ends in angular positions which differ by 180°.

The embodiments typically available for this and their variations are illustrated schematically in FIGS. 4 to 6.

FIG. 4A shows a control element 40 for the control device 34 according to the invention, with which eight force transferring longitudinal elements 42 are arranged in a helical shape over their entire length and are secured to proximal and distal annular collars 44, 46 with an offset of 180°.

With respect to the fact that the diameter of typical control elements is only a few millimeters, on the other hand the required length of the control elements is 10 cm or considerably more, the angles, at which the longitudinal elements arranged in a helical shape deviate from the longitudinal direction of the control elements, are considerably smaller than is perhaps suggested in FIGS. 4 to 6, respectively. In order to clarify this better, two numerical examples are presented here:

In the case of an instrument typically used in neurosurgery, the length of the control device is approximately 30 cm; the length of the associated control element 40 is, therefore, likewise 30 cm. The outer diameter of the control element 40 is typically 1.7 mm. If an angular offset of 180° is selected, at which the proximal and distal ends of the force transferring longitudinal elements 42 are secured to the annular collars 44, 46, a helical line shape of the longitudinal elements results, with which the helical line is inclined relative to the longitudinal axis of the element at an angle of approximately 0.5°.

In the case of an instrument used in laparoscopy, the control device has a length of, for example, 22 cm which corresponds to the length of the control element 40. The outer diameter of the control element 40 is relatively large and is approximately 9.7 mm. With this shorter length of the control device 10 with, at the same time, a considerably larger diameter, an angle of 3.9° is obtained, at which the helical line, along which the force transferring longitudinal elements 42 are arranged, is inclined relative to the longitudinal axis of the control element 40.

The two examples described above can be understood as extreme examples and in the case of the vast majority of control devices 10 according to the invention the angles of inclination of the longitudinal elements 42 relative to the longitudinal axis of the control element 40 will be kept within the limits indicated in these examples.

FIG. 4B shows an alternative embodiment as control element 40′ which is produced from a one-piece small tube 41, for example by way of laser beam cutting.

The slits 43 formed in the small tube 41 by way of laser beam cutting run almost over the entire length of the tube 41 and so annular collars 44′, 46′, which are not slit and which connect the wall segments 45 acting as force transferring longitudinal elements respectively to one another, remain only at the proximal and distal ends.

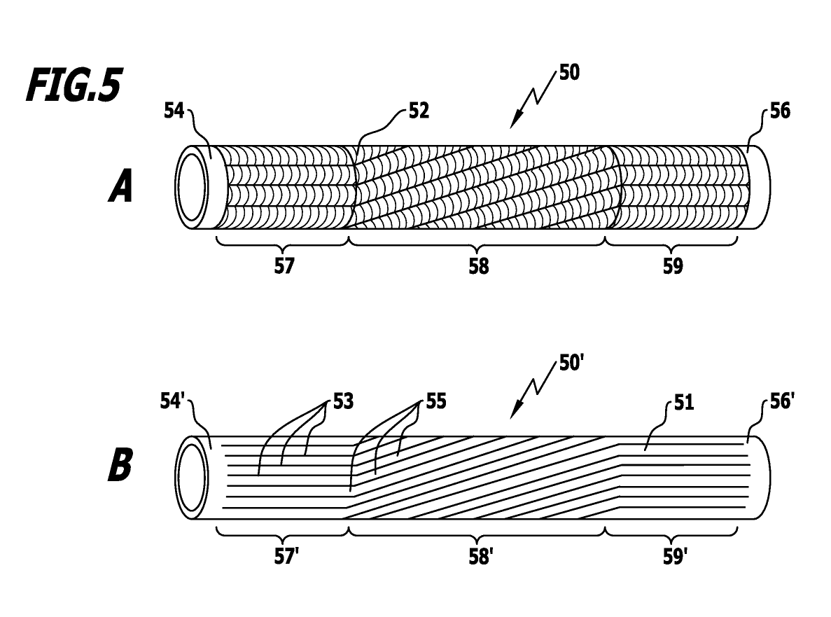

FIG. 5A shows an alternative embodiment to the control element 40 according to the invention in the form of a control element 50, with which eight longitudinal elements 52 are secured in proximal and distal annular collars 54, 56, respectively, wherein, on the other hand, an angular offset in the positioning of the proximal end in relation to the distal end of 180° is present. The longitudinal elements 52 are divided into three different sections, wherein the first section 57 is arranged adjacent to the proximal annular collar 54 and comprises sections of the longitudinal element 52 aligned parallel to the longitudinal direction of the control element 52.

Accordingly, a region of the longitudinal elements 52 is likewise arranged parallel to the longitudinal direction of the control element 50 in a section 59 adjoining the distal annular collar 56.

In the section 58 located therebetween, the remaining regions of the longitudinal elements extending between the sections 57 and 59 extend along helical lines, wherein, in this case, the helical lines are inclined at a somewhat larger angle relative to the longitudinal direction of the control element 50 than is the case in the embodiment of FIG. 4 and so an angular offset of the ends of the respective longitudinal elements, which are secured to the annular collars 54, 56, of 180° can likewise be achieved over a shorter distance.

Even with this example, with which only approximately 50% of the length of the control element is available for the central section, the angles, at which the helical lines are inclined in relation to the longitudinal direction of the control element 50, remain at very small values.

Analogously to FIG. 4B, FIG. 5B shows an alternative embodiment of a control element 50′ which is produced from a one-piece small tube 51, for example by way of laser beam cutting.

The slits 53 formed in the tube 51 by way of laser beam cutting extend almost over the entire length of the tube 51 and so annular collars 54′, 56′, which are not slit and which connect the wall segments 55 acting as force transferring longitudinal elements respectively to one another, remain only at the proximal and distal ends.

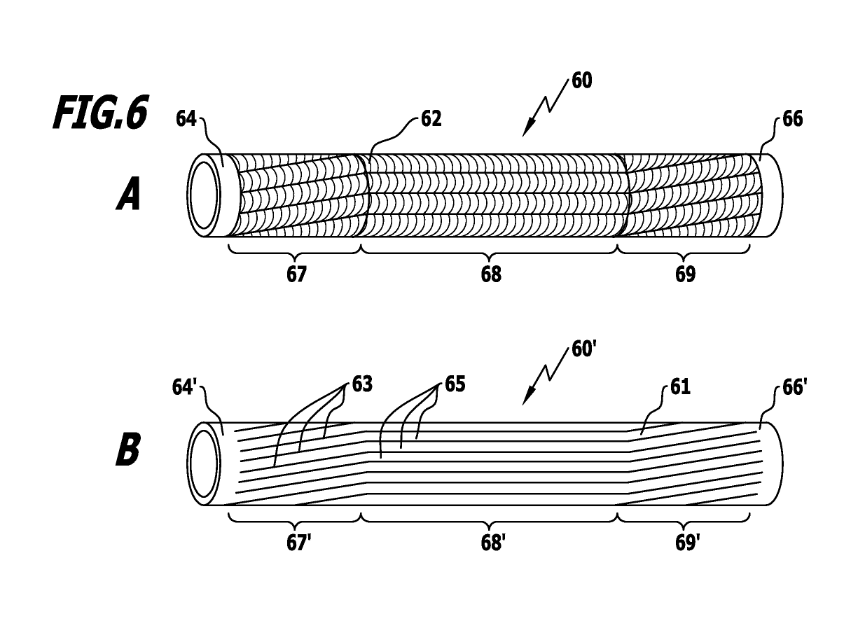

A further variation is shown, finally, in FIG. 6, with which a control element 60 comprises eight longitudinal elements 62 which are secured at an angular offset of 180° to proximal and distal annular collars 64, 66, respectively.

In order to achieve the angular offset, the longitudinal elements are divided into three sections, wherein the respective end sections 67 and 69, i.e. those connected to the annular collars 64 ad 66, respectively, are arranged so as to follow a helical line whereas the regions 68 located therebetween are arranged parallel to the longitudinal axis of the control element 60.

It holds true in this case, as well, in comparison with the embodiment in FIG. 4, that the angle, at which the sections of the longitudinal elements following the shape of a helical line are inclined in relation to the longitudinal direction, is somewhat larger but this can still count as a very small angle.

If an offset other than the 180°, which have been described above on the basis of FIGS. 3 to 6, is selected, a direction of movement for the distal end 38 which deviates from FIG. 3 is obtained; for example, at an offset of 90° any bending of the proximal section 36 in the plane of the paper leads to a deflection of the distal end 38 at right angles out of the plane of the paper.

Preferably, the control elements for the control devices according to the invention can be replaced and so a control device 34 can be given different movement geometries simply by replacing the control element.

Analogously to FIGS. 4B and 5B, FIG. 6B shows an alternative embodiment of a control element 60′ which is produced from a one-piece small tube 61, for example by way of laser beam cutting.

The slits 63 formed in the tube 61 by laser beam cutting extend almost over the entire length of the tube 61 so that annular collars 64′, 66′, which are not slit and connect the wall segments 65 which function as force transferring longitudinal elements respectively to one another, remain only at the proximal and distal ends.

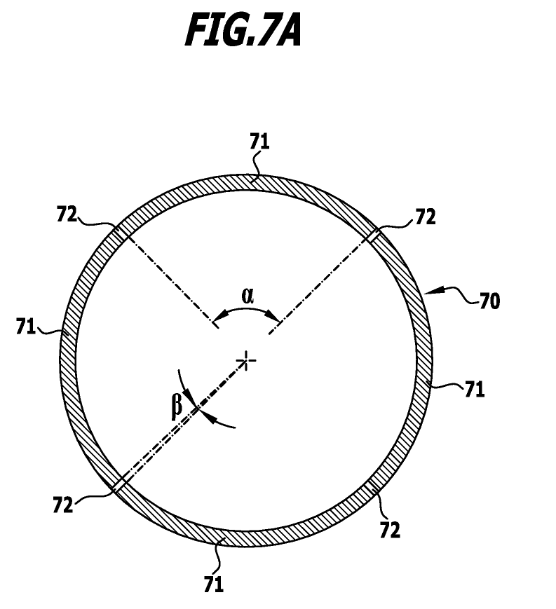

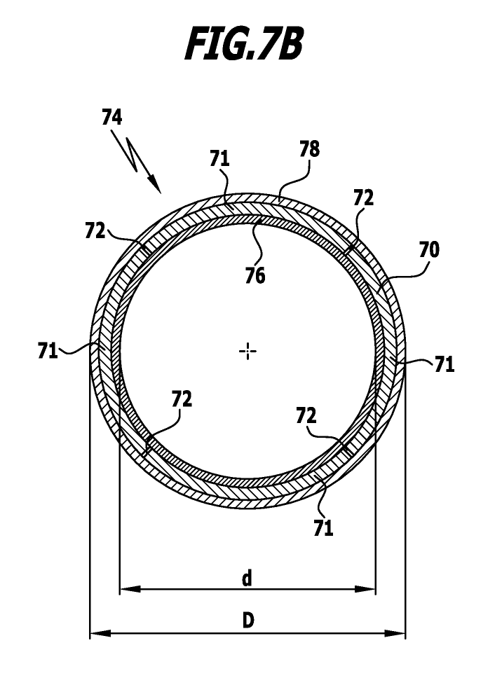

FIG. 7A shows a cross section through a control element 70 analogously to FIGS. 4B, 5B and 6B, with which, however, only four wall segments 71 are present. The arced segments of the wall segments 71 correspond to an arc angle α of approximately 82° to 86°. The extension of the slits 72 in circumferential direction corresponds to an angle β of approximately 4° to 8°.

FIG. 7B shows the cross section of a control device 74, wherein the control element 70 of FIG. 7A is used as control element, with a number of four wall segments 71. The wall segments 71 are spaced from one another via the slits 72.

An outer diameter D of approximately 2.5 mm and an inner diameter of approximately 1.8 mm for the control device 74 are specified by way of example.

The control element 70 is guided at its inner surface by an inner shaft 76 and at its outer surface by an outer shaft 78.

The configuration of the sections of articulation of the control device 34 or 70 has not been mentioned in greater detail. It can be diverse in the form of the flexible sections of the inner and outer shafts 76, 78, respectively.

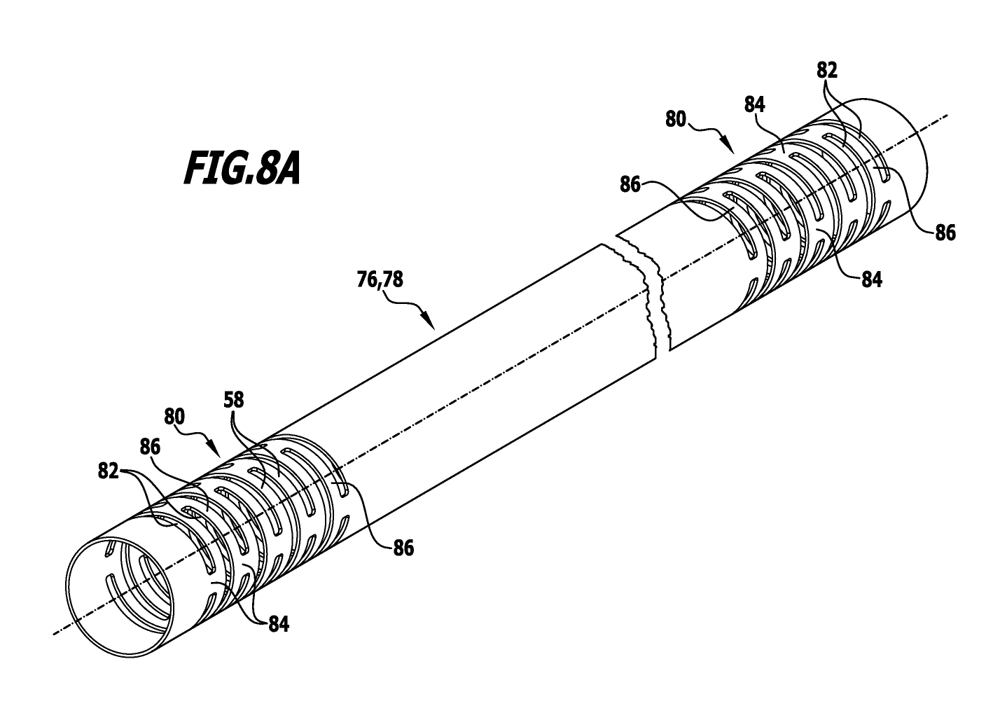

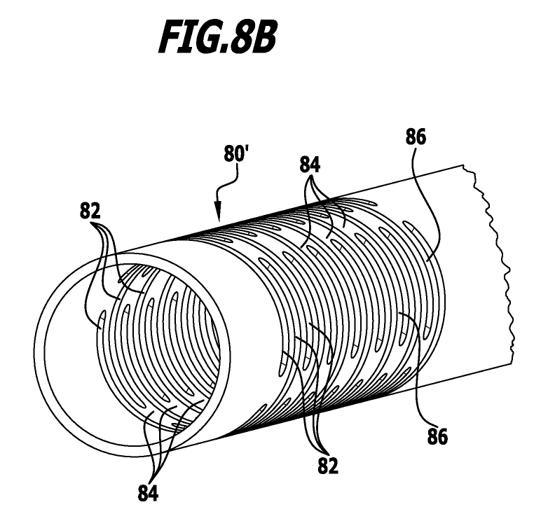

FIGS. 8A and 8B show two variations of related configurations of the flexible sections, here in the form of the sections 80 and 80′, respectively.

The two variations have in common the use of a slit structure with slits 82 extending in circumferential direction in the hollow cylindrical shaft. Preferably, two or more slits which are separated from one another via webs 84 are present along a circumferential line. Since the arrangement of slits along only one circumferential line would allow only a very small pivoting angle, a plurality of circumferential lines with slits 82, spaced in axial direction via webs 86, are present in typical slit structures of the areas of articulation 80, 80′. Slits 82 arranged adjacent to one another in axial direction are preferably arranged so as to be offset relative to one another in circumferential direction so that bending possibilities in several planes result.

In FIG. 8A, two slits 82, which are separated from one another by webs 84, are present per circumferential line. In FIG. 8B, there are three slits 82. The slit structure typically comprises in both cases a plurality of slits 82 which are arranged along several imaginary circumferential lines which are spaced from one another in axial direction via webs 86. The admissible pivoting angle may be predetermined very easily via the selection of the slit structure and the number of slits and also additional properties of a section of articulation, such as, for example, the bending strength, can be adapted to the respective application.

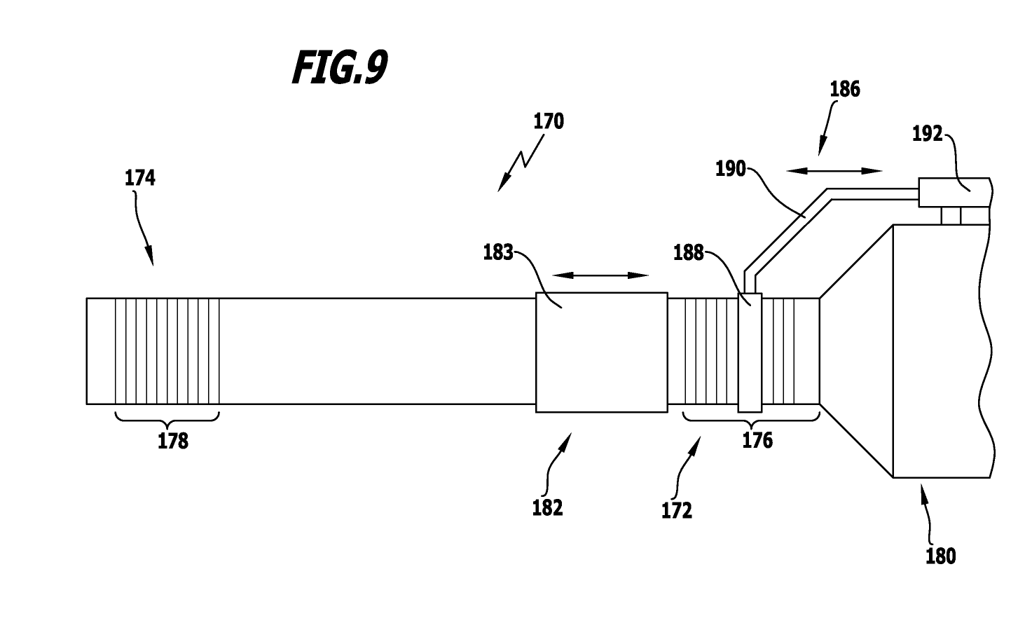

Finally, FIG. 9 shows the present invention in a further variation with a control device 170 with a proximal end section 172 and a distal end section 174 with respectively associated areas of articulation 176 and 178.

A handling device 180 is connected to the proximal end section 172 of the control device 170.

The areas of articulation 176 and 178 are designed with essentially the same length so that when the proximal end section 172 is bent through, for example, 30°, a corresponding angling of the distal end section 174, likewise through 30°, results. The direction, in which the angling of the distal end section 174 takes place, depends on the selection of the control element which is not shown here in detail and the securing in position of the ends of the force transferring longitudinal elements, as described above in detail.

The control device 170 shown in FIG. 9 has, in addition, a holding device 182 in the form of a sleeve 183 which is arranged on the outer shaft of the control device 170 so as to be displaceable longitudinally.

If the sleeve 183 is displaced in the direction towards the proximal end section 172 and if the sleeve 183 is allowed to overlap with the area of articulation 176, the area of articulation 176 is shortened, whereby its maximum bending angle is restricted. As a result, the admissible bending angle in the region of the distal end section 174 may be variably adjusted so that, for example, a defined working area can be adjusted under the view of the operator during the endoscopic removal of pathological structures.

FIG. 9 contains an alternative solution to the holding device 182 in the form of the holding device 186 which comprises a ring 188 which is secured to the handling device 180 so as to be displaceable longitudinally via a bar 190 with a double elbow and a straight-line guide 192. The part of the area of articulation 176 available for the bending movement of the proximal end section may, as already explained with respect to the sleeve 183, be shortened via the alteration in the position of the ring 188 along the section 176 and so, on the other hand, only a restricted bending angle will be allowed on the side of the distal end section 174.

In addition, it is conceivable, both in the case of the sleeve 183 and in the case of the ring 188, for them to be securable in a predetermined position, i.e. with a predetermined overlapping of the area of articulation, so that the adjusted, restricted working area on the side of the distal end section 174 is ensured.

On the other hand, it is conceivable to displace the sleeve 183 in the direction of the distal section of articulation 178, as well, wherein a converted, i.e. stronger pivoting movement will then take place in the region of the distal end section 174 with a corresponding pivoting movement of the proximal end section 172.

It is likewise conceivable to provide markings for the position of the sleeve 183 and the ring 188, respectively, or its straight-line guide 192 so that an angular restriction once found can also be adjusted exactly at a later time and repeatedly.

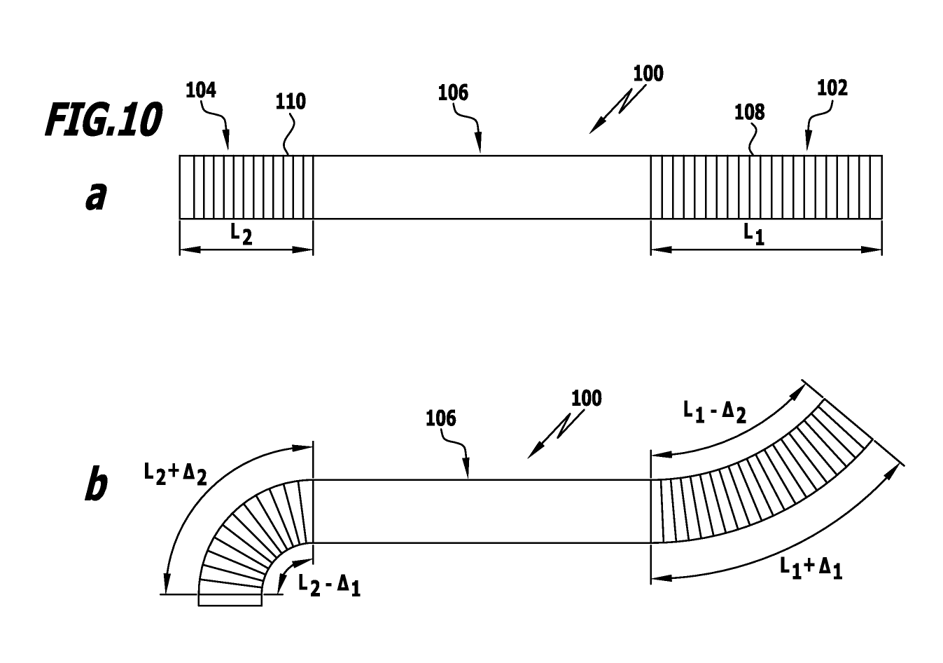

In order to explain the effect described above of the amplification of the pivoting or bending movement at the distal end, reference is made to FIG. 10 which shows a control device 100 which has a proximal end section 102, a distal end section 104 as well as a central section 106 located therebetween. Whereas the central section 106 is designed to be flexurally rigid, the proximal and distal end sections 102, 104 each contain an area of articulation 108 and 110, respectively, with a length L₁ and L₂, respectively, measured in axial direction. The length L₂ is selected to be shorter than the length L₁. FIG. 8A shows the control device 100 in the basic position, in which no forces act on the proximal end section 102.

If the proximal end section 102 is pivoted out of the axial direction, as clearly shown in the illustration of FIG. 10b, an increased length of the area of articulation 108 of L₁+Δ₁ results in the proximal area of articulation 108 at the outer radius of the bent end area 102, a shortened length of L₁−Δ₂ results at the inner radius. Corresponding changes in the lengths result for the distal end section 104 with a length at the outer radius of L₂+Δ₂ and a length at the inner radius of L₂−Δ₁. Since the lengths L₁ and L₂ of the areas of articulation 108, 110 are different, an amplified bending movement results automatically for the distal end section 104 in order to be able to follow the changes in length predetermined by the proximal end section.

This effect may also be utilized to make complete use of the distal pivoting radius possible, for example, in a proximally limited working area with relatively small pivoting movements and to provide as large a working area as possible distally.

This principle may be used in a variable manner with the present invention in that the length of one area of articulation will be varied in relation to the other one via a holding device (cf. FIG. 9).

1. Control device, in particular for use in endoscopes or the like, comprising a proximal and a distal end section each comprising an area of articulation as well as a central section arranged therebetween, with an outer hollow cylindrical shaft, an inner hollow cylindrical shaft as well as a control element arranged between these shafts and having two or more longitudinal elements extending substantially from the proximal to the distal end section of the control device and transferring force,
wherein the longitudinal elements are arranged at essentially regular angular distances in circumferential direction of the control device and are connected to one another in circumferential direction in the region of their respective proximal and distal ends, wherein the distal ends of the longitudinal elements are secured in circumferential direction in angular positions differing from the angular positions, in which the respectively associated proximal ends are secured.

2. Control device as defined in claim 1, wherein the angular positions differ in circumferential direction by approximately 10° to 360°, in particular by approximately 45° to 315°.

3. Control device as defined in claim 1, wherein the force transferring longitudinal elements are arranged so as to be laterally spaced from one another.

4. Control device as defined in claim 3, wherein spacer elements are arranged between the force transferring longitudinal elements.

5. Control device as defined in claim 1, wherein the force transferring longitudinal elements are arranged along the longitudinal direction at least partially in direct contact with one another.

6. Control device as defined in claim 1, wherein the force transferring longitudinal elements are guided in a radial direction by the outer and the inner shaft.

7. Control device as defined in claim 1, wherein the force transferring longitudinal elements are arranged in a helical shape between the shafts at least over part of their length.

8. Control device as defined in claim 7, wherein the force transferring longitudinal elements are arranged essentially parallel to the longitudinal direction of the control device in the region of the proximal and/or distal ends and in a helical shape in a region located therebetween.

9. Control device as defined in claim 7, wherein in the region between their proximal and distal ends the force transferring longitudinal elements have one or more sections arranged parallel to the longitudinal direction of the control device.

10. Control device as defined in claim 1, wherein the force transferring longitudinal elements are designed as cables or wires.

11. Control device as defined in claim 1, wherein the force transferring longitudinal elements have a banana-shaped cross section.

12. Control device as defined in claim 1, wherein the control element comprises a hollow cylindrical component, the cylinder wall thereof being subdivided at least in the region of a section between the proximal and distal ends into two or more wall segments forming the force transferring longitudinal elements.

13. Control device as defined in claim 12, wherein the two or more wall segments are connected fixedly to one another via an annular collar at the distal end of the hollow cylindrical component.

14. Control device as defined in claim 12, wherein the two or more wall segments are connected fixedly to one another in the region of the proximal end of the hollow cylindrical component.

15. Control device as defined in claim 1, wherein the outer and/or the inner shaft has a proximal and a distal section of articulation in the region of the proximal and distal areas of articulation of the control device.

16. Control device as defined in claim 15, wherein at least one of the outer and inner shafts has a flexurally rigid section arranged between the proximal and distal areas of articulation.

17. Control device as defined in claim 16, wherein the proximal area of articulation has an extension in longitudinal direction of the control device differing from the extension of the distal area of articulation.

18. Control device as defined in claim 17, wherein the extension of the proximal and/or the distal area of articulation is adjustable.

19. Control device as defined in claim 18, wherein the control device comprises a holding device for fixing in position parts of one area of articulation in a flexurally rigid manner with respect to the central section of the control device or a functional unit adjoining its proximal or distal end section.

20. Control device as defined in claim 1, wherein at least one of the areas of articulation is designed to be elastic.

21. Control device as defined in claim 1, wherein the area(s) of articulation of the outer and/or inner shaft comprise a wall section, several slits spaced from one another and extending in circumferential direction being arranged in said wall section.

22. Control device as defined in claim 21, wherein two or more, in particular three or more slits are arranged one behind the other in circumferential direction.

23. Control device as defined in claim 21, wherein three or more slits are arranged next to one another in axial direction.

24. Control device as defined in claim 23, wherein the slits arranged next to one another are arranged so as to be offset relative to one another in circumferential direction.

25. Control device as defined in claim 21, wherein the slits are slits penetrating the cylinder wall completely.

26. Control device as defined in claim 21, wherein the wall surfaces delimiting the slits are arranged at an acute angle in relation to the radial direction.

27. Control device as defined in claim 26, wherein wall surfaces of the same slit located opposite one another are arranged in mirror image so that a larger slit width results at the outer circumference of a shaft than adjacent to the inner circumference.
